(19) United States
(12) Patent Application Publication
(10) Pub. No.: US 2015/0219632 A1
(43) Pub. Date: Aug. 6, 2015

(54) METHOD AND APPARATUS FOR SYSTEMATIC SINGLE CELL TRACKING OF DISTINCTIVE CELLULAR EVENTS

(71) Applicants: Masahiko Sato, Quebec (CA); Sachiko Sato, Quebec City (CA)

(72) Inventors: Masahiko Sato, Quebec (CA); Sachiko Sato, Quebec City (CA)

(21) Appl. No.: 14/680,641

(22) Filed: Apr. 7, 2015

Related U.S. Application Data
(62) Division of application No. 13/880,793, filed on Apr. 22, 2013.
(60) Provisional application No. 61/406,362, filed on Oct. 25, 2010.

(51) Int. Cl. G01N 33/50

(52) U.S. Cl. CPC G01N 33/5091; G01N 33/5017

(57) ABSTRACT

A method and apparatus for quantitative identification of distinctive cellular events occurring in a cell population using a non-fluorescence approach. The apparatus and method comprises an image acquisition unit having a Differential Interference Contrast microscope with a camera, a light source, an environmental chamber allowing carrying out cell culture of at least one cell in a cell population; the image acquisition unit acquiring images of the cell population, at predetermined time points; a cell tracker for individually tracking the at least one cell of the cell population in the images; a distinctive cellular event detector for detecting an occurrence of a distinctive cellular event; a report generator; wherein the distinctive cellular event is selected from the group consisting of: tripolar, tetrapolar, quadpolar cell division, cell fusion, cell death, impaired cell division, cell shape alteration, nuclear shape alteration, inner cellular material accumulation, cell enlargement, engulfing, hyper-mobilization, hypo-mobilization and prolonged doubling time.

### CROSS-REFERENCE TO RELATED APPLICATIONS

This application is a divisional application of U.S. application Ser. No. 13/880,793 filed Apr. 22, 2013, which is a 35 U.S.C. §371 national phase application of PCT/IB2011/054436 filed Oct. 7, 2011, the disclosure of which is hereby incorporated by reference. The present application claims the benefit of priority under 35 U.S.C. §119(e) of U.S. provisional patent application No. 61/406,362 filed Oct. 25, 2010, the disclosure of which is hereby incorporated by reference.

### TECHNICAL FIELD

The invention relates to the single cell quantitative identification of rare distinctive cellular events occurring in a minority of cells of a population using a non-fluorescence approach.

### BACKGROUND OF THE ART

Many diseases, e.g. cancer, could arise from a single or a handful of distinctive cells that could be found within a large number of normal cells.

In the human body, distinctive or malignantly transformed cells could be created by exposure to carcinogenic substances that contaminate the environment. Such distinctive or malignantly transformed cells could also be found in the fluids or blood of humans who have taken a substance. It thus should be apparent that laboratory techniques for the study of human cancer or carcinogens need to have the ability to precisely pin-point the moment of formation of distinctive cell, malignant transformation or the initiation of the cascade that leads to malignant transformation or diseases.

However, none of the currently existing in vitro genotoxicity and mutagenicity tests have readily allowed such investigations since concentrations of carcinogens in the environment are generally too low to induce any cell responses in the majority of cells. In other words, currently available tests are only optimal to detect major and frequent events induced by a substance and not sensitive to sense the rare events induced by doses which humans are anticipated to be exposed to.

As an experimental compromise and to induce detectable levels of responses with existing techniques, high doses of substances are used in those tests for the identification or screening of human carcinogens. Then, these laboratory studies therefore have to rely on high-to-low dose extrapolation to gain insight into the mechanisms of malignant transformation and identification of possible human carcinogens, or any type of initiation of diseases although this extrapolation is known to be inaccurate.

The existing methods or tests are optimal to detect or analyze the commonalities of the responses among cells. Thus, these methods could miss important information, because they ignore the individuality, which is related to the distinctiveness found in the handful of altered cells or the events that only occur to those “index case” cells. Inaccuracy or limitation associated with existing methods to investigate the mechanisms of malignant transformation, human carcinogen identification or the origin of many other human diseases is therefore most likely related to the underling concept or design of existing methods.

At the cell culture level, the environmental doses of carcinogen would not show any detectable effects on e.g. cell growth rate, frequency of cell death induction and doubling time of cells, while these doses of carcinogens could still induce distinctiveness in a small number of cells since these doses indeed induce cancers in humans.

### SUMMARY

The present invention is designed to search and quantitatively identify distinctive cells, distinctive cellular events and cells that show distinctive behavior from other cells within a cell population regardless of the frequency or dominance, by using single cell tracking. This invention is carried out by long-term live cell imaging, individual cell tracking, cell lineage database creation and quantitative analysis.

Because excitation of fluorophore by relevant wavelength of light causes cytotoxicity, non-florescence imaging is used and, because all cellular events are recorded in chronological order, distinctive cellular events occurring as rare as once in a 160 hrs period within 16,000 cells can be detected.

Cells are cultured on the microscope stage in order to create live cell imaging movies. These movies are then used for individual cell tracking. A database of these movies is then created for analysis and the nature and quantity of cellular events are determined and collected in order to categorize the response of such cell or cell population following exposure to a test agent, in one embodiment, at low dose (e.g. non-cytotoxic dose or environmental dose).

Therefore, in a first embodiment, the present invention relates to a method for identifying distinctive cellular events occurred within normal events during cell culture under testing conditions, comprising the steps of: maintaining a cell culture under microscopic observation; gathering time lapse data by capturing sequential images during observation of at least one progenitor for a sufficient time to allow division of the progenitor to form at least one progeny under normal conditions; using time lapse data at Time point=1 for indexing at least one progenitor of the culture with a unique identifier; using time lapse data for identifying at least one progeny stemming from the progenitor to establish a lineage for the indexed progenitor; identifying at least one cellular event associated with the lineage; categorizing the cellular event to establish an event category; recording number of the event category associated with the lineage.

The occurrence and/or number of categorized cellular event could correlate with a reaction of the cell lineage to the testing conditions.

The method can be carried out regardless of frequency or dominance of occurrence of the distinctive cellular events within the normal events.

In a second embodiment, the present invention relates to a method for evaluating increased risk of occurrence of distinctive cellular events upon exposure to a test agent or a treatment, comprising the steps as described above and further comprising the step of: comparing occurrence and/or number of at least one of the event category of the test culture with a control culture devoid of the agent or the treatment; wherein a higher number of at least one distinctive event category in the treated culture compared to control culture is an indication that the agent has an effect to induce distinctiveness or unique/distinctive cell behaviors into a number of cells on the cell culture.

In one embodiment, with respect to the first and second embodiments of the invention, the event category is selected from: normal or distinctive cellular events; presence, absence or level of a cell marker.

In a further aspect of the invention, the cellular events are selected from the group consisting of: no division, dipolar division (DD), tripolar division (TD), quadripolar division (QD), hexapolar division (HD), cell fusion (CF), cell death (CD), incomplete or partial division (IP), cell shape alteration, nuclear shape alteration, inner cellular material accumulation, cell enlargement, engulfing, hyper-mobilization, hypo-mobilization, and prolonged doubling time. New event types, which are currently not identified or not yet be classified, could be added.

In a third embodiment, the invention relates to a method for the identification of an “index case” cell and the dose of a potential test agent causing it, the method comprising the steps of: maintaining a cell culture in the presence of a non-cytotoxic dose of a test agent under microscopic observation; capturing sequential images during incubation of at least one progenitor for a sufficient time to allow division of the progenitor to form at least one progeny; using sequential images at Time point=1 for indexing at least one progenitor with a unique identifier; using sequential images for identifying at least one progeny stemming from the indexed progenitor to establish a lineage; identifying at least one distinctive cellular event associated with the lineage; recording number of the distinctive cellular events associated with the lineage; wherein the presence of a distinctive cellular event associated with at least one cell lineage is an indication that the agent is a potential candidate to induce distinctiveness or unique cell behaviors into small number of cells on the cell culture.

In one embodiment, with respect to the aforementioned embodiments, the test agent may be an agent leading to distinctive cellular events by its non-cytotoxic dose of distinctive cellular event inducer (referred to herein as DCEI).

In one embodiment, the invention provides a method for the identification of a potential DCEI and the method comprising the steps of:—maintaining a cell culture in the presence of a non-cytotoxic dose of the agent under microscopic observation;—capturing sequential images during observation of at least one progenitor for a sufficient time to allow division of the progenitor to form at least one progeny;—using sequential images at Time Point=1 for indexing at least one progenitor with a unique identifier;—using sequential images for identifying at least one progeny stemming from the indexed progenitor to establish a lineage;—identifying at least one distinctive cellular event associated with the lineage; and —recording number of the distinctive cellular events associated with the lineage; wherein the at least one distinctive cellular event is selected from the group consisting of: TD, QD, HD, CF, IP, cell shape alteration, nuclear shape alteration, inner cellular material accumulation, cell enlargement, engulfing, hyper-mobilization, hypo-mobilization, prolonged doubling time, and new event types, which are currently not identified or not yet be classified; whereby the presence of a distinctive cellular event associated with at least one cell lineage is an indication that the agent is a potential DCEI.

In one embodiment, with respect to the invention, the method provides a means to screen for DCEI in a large number and variety of potential carcinogenic, genotoxic, mutagenic and cytotoxic agents. Also part of the potential DCEI may be infectious agents such as bacteria or viruses or even inflammation agents such as urea crystals, aggregated proteins or prions, or biologically active natural products or genetic materials.

In one embodiment, DCEI may be a cytotoxic compound, or tumor promoter, or radiation such as ultraviolet light, infrared, electromagnetic field, microwaves; or particles and materials, such as asbestos fibers; or chemical substances or particulate matter found in exhaust gas or in cigarette smoke.

In one embodiment, the method provides a means to establish, which agents may act as potential DCEI under chronic exposure conditions (i.e. long incubation period) at non-cytotoxic dose.

In one embodiment, with respect to the invention, the method provides a means to observe timing of distinctive cellular event, infection, timing of uptake of materials by cells or timing of cell-cell (pathogen) contact.

In one embodiment, the method of the invention also provides assays to test substances that would inhibit or prevent or counteract the DCEI once this DCEI has been identified in the method of the present invention.

In one embodiment, the present method may allow one to distinguish between anti-cancer drugs that are mutagenic or may produce cytotoxic side effects from anti-cancer drugs that are safer for administration.

In one embodiment, with respect to the third embodiment of the invention, the distinctive cellular event is selected from: distinctive cellular events (such as TD, QD, HD, CF, IP, cell shape alteration, nuclear shape alteration, inner cellular material accumulation, cell enlargement, engulfing, hyper-mobilization, hypo-mobilization or prolonged doubling time) or change in the level of a cell marker from its normal state or new event types, which are currently not identified or not yet be classified.

In one embodiment, the change in level of a cell marker may be assessed qualitatively by observing the presence of a cell marker normally absent in or on the cell, or observing the absence of a cell marker normally present in or on the cell.

In a further aspect of the invention, the distinctive cellular events may be observed when observing distinctive cellular events, which are selected from the group consisting of: TD, QD, HD, CF, CD, IP, cell shape alteration, nuclear shape alteration, inner cellular material accumulation, cell enlargement, engulfing, hyper-mobilization, hypo-mobilization and prolonged doubling time, and new event types, which are currently not identified or not yet be classified.

In a further aspect of the invention, the dose of the DCEI causing distinctive cellular event is determined upon testing different doses and establishing the first concentration providing a first “index case” cell presenting an event that may be associated with distinctiveness in its lineage. Such distinctive cellular event may be selected from: TD, QD, HD, CF, IP, cell shape alteration, nuclear shape alteration, inner cellular material accumulation, cell enlargement, engulfing, hyper-mobilization, hypo-mobilization, prolonged doubling time, and new event types, which are currently not identified or not yet be classified.

In a further aspect of the invention, the step of capturing the sequential images is carried out with a camera mounted on a microscope, wherein cells are cultured on the stage of microscope in a confined and stationary manner under controlled conditions. In one embodiment, cells are incubated in a multiwell chamber. In some cases, the cells can be incubated under testing conditions concurrently with a culture under control conditions in a different well of the chamber, such as to ensure proper control for comparison purposes.

In a further aspect of the invention, the cells are incubated under observation for any length of time to detect distinctive cellular events. In this respect, the cells are observed under microscope without the use of fluorescence to avoid the presence of phototoxicity derived from excitation and emission of fluorophores since this may cause deleterious effects on the control conditions. In one embodiment, the cells are observed under microscope by using a differential interference contrast (DIC).

In one embodiment, the cells cultured are eukaryotic cells. In one embodiment, the cells are mammalian cells, for example human cells.

In one embodiment, the cells are adherent cells. Still, in one embodiment, the cells are from an immortalized cell line or from cultured cells from a fresh tissue sample or any cells that can be maintained in a well.

In accordance with another aspect, the method described herein is provided as a computer-implemented method. In particular, once the cells under observation have been recorded for a given number of hours, cell evolution is tracked in an automated manner, which leads to the generation of lineage maps and comparisons of compiled data to normal and distinctive cell behavior. The lineage maps and comparisons may also be part of the computer-implemented method such that they are performed in an automated manner.

Therefore, there is described herein a computer-implemented method for identifying occurrence of rare distinctive cellular events during cell culture under testing conditions, and a system for identifying occurrence of rare cellular events during cell culture under testing conditions. The system is composed of a combination of hardware and software elements, which cooperate together to implement the computer-implemented method. The software components may be provided in a single application or a combination of two or more applications coupled to a processor.

According to one broad aspect there is provided an apparatus for quantitative identification of distinctive cellular events occurring in a cell population using a non-fluorescence approach. The apparatus comprises an image acquisition unit having a Differential Interference Contrast (DIC) microscope system with a camera, a light source, an environmental chamber allowing carrying out cell culture of at least one cell in a cell population, for a period of time sufficient to allow division of a progenitor to form at least one progeny; a controller for controlling the image acquisition unit to acquire images of the cell population in the chamber, at predetermined time points; a cell tracker for individually tracking the at least one cell of the cell population in the images acquired by the image acquisition unit; a distinctive cellular event detector for detecting an occurrence of a distinctive cellular event for at least one of the cell individually tracked by the cell tracker; a report generator for generating a report having an identification of detected distinctive cellular events; wherein the distinctive cellular event is selected from the group consisting of: tripolar cell division (TD), tetrapolar cell division (QD), quadpolar cell division (HD), cell fusion (CF), cell death (CD), impaired cell division (IP), cell shape alteration, nuclear shape alteration, inner cellular material accumulation, cell enlargement, engulfing, hyper-mobilization, hypo-mobilization and prolonged doubling time.

In one embodiment, the light source emits light at a wavelength of one of visible light and near infrared light.

In one embodiment, the period of time is sufficient to allow multiple generations of progenies to be produced by the at least one cell.

In one embodiment, the period of time is between 7 minutes and 200 hours.

In one embodiment, the number of the at least one cell in the cell population is greater than 100 at a beginning of the period of time.

In one embodiment, the microscope system comprises a X-Y stage and a Z-drive controlled by the controller.

In one embodiment, the image acquisition unit further has at least one of an optical filter, a high magnification objective and a coupler.

In one embodiment, the apparatus further comprises an image file arranger to organize the images generated by the image acquisition unit.

In one embodiment, the chamber is divided in at least two fields of view, wherein the controller controls the image acquisition unit to generate image files from each of the fields of views, the image arranger organizing the images generated by the image acquisition unit in a set for each of the at least two fields of view.

In one embodiment, the cell tracker tracks each and all of the at least one cell of the cell population individually.

In one embodiment, the apparatus further comprises a cell lineage tracker for assigning unique identifiers for the at least one cell individually tracked by the cell tracker.

In one embodiment, the distinctive cellular events occur in less than 10% of the cell population.

In one embodiment, the apparatus further comprises an event storage unit for indexing the distinctive cellular events in association with the individual cells tracked.

In one embodiment, the report generator includes lineage information in the report.

In one embodiment, the chamber is treated with a treatment.

In one embodiment, the chamber is a multiwell culture chamber with at least 2 wells.

According to another broad aspect, there is provided a method for quantitative identification of distinctive cellular events occurring in a cell population using a non-fluorescence approach. The method comprises providing an image acquisition unit having a Differential Interference Contrast (DIC) microscope system with a camera, a light source, an environmental chamber allowing carrying out cell culture of at least one cell in a cell population, for a period of time sufficient to allow division of a progenitor to form at least one progeny; controlling the image acquisition unit to acquire images of the cell population in the chamber, at predetermined time points; individually tracking the at least one cell of the cell population in the images acquired by the image acquisition unit; detecting an occurrence of a distinctive cellular event for at least one of the cell individually tracked by the cell tracker; generating a report having an identification of detected distinctive cellular events; wherein the distinctive cellular event is selected from the group consisting of: tripolar cell division (TD), tetrapolar cell division (QD), quadpolar cell division (HD), cell fusion (CF), cell death (CD), impaired cell division (IP), cell shape alteration, nuclear shape alteration, inner cellular material accumulation, cell enlargement, engulfing, hyper-mobilization, hypo-mobilization and prolonged doubling time.

According to another broad aspect, there is described a use of the apparatus for quantitative identification of rare distinctive cellular events occurring in a cell population using a non-fluorescence approach to detect an individuality of cells in the cell population.

In one embodiment, the cell population is treated with a non-cytotoxic dose of a substance.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art.

In this specification, the expression “distinctive cellular events” is intended to mean the events which do not occur during the regular cell growing process to produce two progenies. Such events include multipolar cell division (including tripolar cell division (TD), tetrapolar cell division (QD), quadpolar cell division (HD)), impaired cell division (IP), cell fusion (CF), cell death (CD), cell shape alteration, nuclear shape alteration, inner cellular material accumulation, cell enlargement, engulfing, hyper-mobilization, hypo-mobilization, and prolonged doubling time.

In this specification, the expression “non-cytotoxic dose” is intended to mean the dose, of which exposure does not lead to the prolongation of cell doubling time, to the reduction of cell growth rate, and to the increased frequency for the formation of dead cells. For example, a non-cytoxic dose means a dose that induces less than 5% cell death in an endpoint assay when compared to the control, most particularly it can induce less than 1% cell death, less than 0.1% cell death or even less than 0.01% cell death.

In this specification, the expression “distinctive cellular event inducer (referred to herein as DCEI)” is intended to mean a substance that induces distinctive cellular events at their non-cytotoxic dose.

The term “rare cellular event” means cellular events, which are different from normal events. Multipolar cell division, cell division suppression and cell fusion for instance. These rare cellular events occur in non-treated cells and in cells treated with non-cytotoxic doses of given agents. In this context, the term rare means events occurring at such a low frequency that it is not evident in a cell population at a macroscopic level. For example, the method of the present invention allows one the capacity to observe or detect distinctive cellular events that occur once in 16,000 cells in 160 hours of observation, or at about 10% frequency or more; 1% frequency or more; 0.1% frequency or more; particularly at about 0.01% or more; more particularly at about 0.001% or more.

The term “index case” means a primary case or cell zero that acquires some altered phenotype in a cell population. The term “index-case” also refers to the first cell that experiences a distinctive cellular event that may lead to distinctive behavior of the resulting cell population such as, for example, tumor cells.

### BRIEF DESCRIPTION OF THE DRAWINGS

Having thus generally described the nature of the invention, reference will now be made to the accompanying drawings, showing by way of illustration a example embodiment thereof and in which:

FIG. 1 is a block diagram showing an example apparatus for analysis of distinctive cellular events;

FIG. 2 is a flow chart of the main steps carried out using the image acquisition unit of the apparatus of FIG. 1;

FIG. 3 is a flow chart of the main steps carried out using the Image quality controller of the apparatus of FIG. 1;

FIG. 4 is a flow chart of the main steps carried out using the movie creator of the apparatus of FIG. 1;

FIG. 5 is a flow chart of the main steps carried out using the live cell events analyzer of the apparatus of FIG. 1;

FIG. 6 is a flow chart of the main steps carried out using the cell lineage creator and data processor of the apparatus of FIG. 1;

FIG. 7 is a flow chart of the main steps carried out using the report generator of the apparatus of FIG. 1;

FIG. 8 includes FIG. 8A, 8B, 8C wherein FIG. 8A is a graphical representation of the apparatus of FIG. 1, FIG. 8B is a graphical representation of the wells of the chambered coverglass, FIG. 8C is a graphical representation of the field-of-views of the wells of the chambered coverglass;

FIG. 9 is a sequence of images extracted from the images taken at different times to show the progression in the quantity of cells present in one field-of-view;

FIG. 10 includes FIG. 10A, 10B, 10C, 10D, 10E, 10F which are photographs showing a typical panorama view, cell lineage number and cell numbering, in FIG. 10A an example of panorama view (T=1) is shown. In FIG. 10B, each progenitor was numbered. Cells that moved out from FOVs were excluded from numbering. A magnified image of the panorama view (T=1) is shown in FIG. 10C. Cell Lineage 41 is indicated in FIG. 10C. An example of panorama view (T=852) is shown in FIG. 10D. All progeny cells were identified and numbered, in FIG. 10E. In the magnified view shown in FIG. 10F, five surviving progenies of cell lineage 41 (41-4, 41-8, 41-10, 41-12 and 41-14) can be found;

FIG. 11 is a graphical representation of a cell lineage diagram based on data found in FIG. 10, this map corresponds to cell lineage 41 indicated in FIG. 10C. During long-term live cell imaging, progenies entered cell death (CD), dipolar division (DD), tripolar division (TD) and cell fusion (CF);

FIG. 12 is a graph of the cell growth curve determined based on cell lineage data and includes FIG. 12A and FIG. 12B, wherein Non-treated cells (FIG. 12A) and cells exposed to 1 μM methylnitronitrosoguanidine (MNNG) (FIG. 12B) were used for individual cell tracking, after entering data into Database, the number of cells in every 10 min was calculated. Means and standard deviations are shown;

FIG. 13 is a graph of the doubling time determined based on cell lineage data and includes FIG. 13A and FIG. 13B, wherein by employing database, doubling time of non-treated cells (FIG. 13A) and cells exposed to 1 μM MNNG (FIG. 13B) was determined, standard deviations are shown;

FIG. 14 includes photographs showing the categorization of dipolar division (DD), tripolar division (TD), quadrupolar division (QD) and impartial mitosis (IP) for HeLa cells;

FIG. 15 includes photographs showing the categorization of cell fusion (CF), in which HeLa cells in T=1 were defined as F0 and first-level progenies and second-level progenies as F1 and F2, respectively, F1M represents progenies that entered mitosis; and

FIG. 16 includes photographs showing the categorization of cell death (CD) for HeLa cells, including all types of CD regardless of observed patterns which depend on cell types.

It will be noted that throughout the appended drawings, like features are identified by like reference numerals.

### DETAILED DESCRIPTION

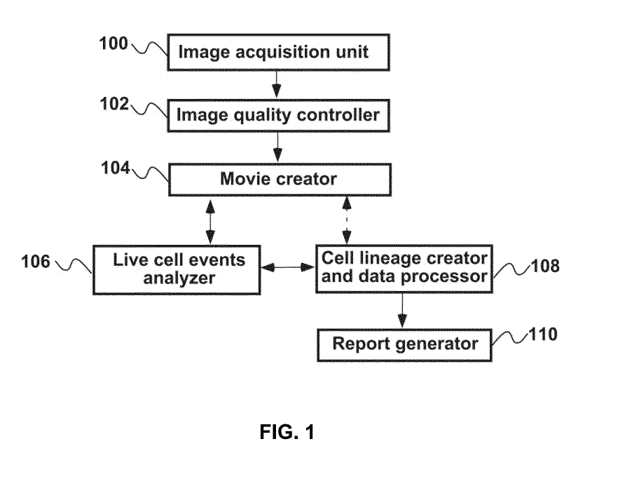

FIG. 1 is a block diagram illustrating an embodiment of an apparatus for analysis of distinctive cellular events, which are induced by non-cytotoxic doses of substances and occur at a low percentage of normal cellular events. For example, this low percentage can be 10% or less. In one example case, the percentage can be 0.001%. Because such events can occur any moment during cell culture, this apparatus creates a cell imaging movie of treated and non-treated cells, and analyzes recorded cells individually.

The Image acquisition unit 100 is composed of a microscope, a CCD camera, a light source, optical filters and elements, a coupler for enlargement of images, an environmental chamber, an adaptor to hold multiwell culture chamber, a X-Y stage and a Z-drive. The light source can be halogen light or LEDs, which create white light or emit some range of visual wavelength such as near infrared. The environmental chamber allows carrying out cell culture on microscope stage for a prolonged period of time, for example for at least 160 hrs at the desired temperature, concentrations of oxygen, carbon dioxide, and nitrogen gas and humidity. The cells are illuminated by a method, which allows visualizing cell structures. The illumination method can be Differential interference contrast (DIC).

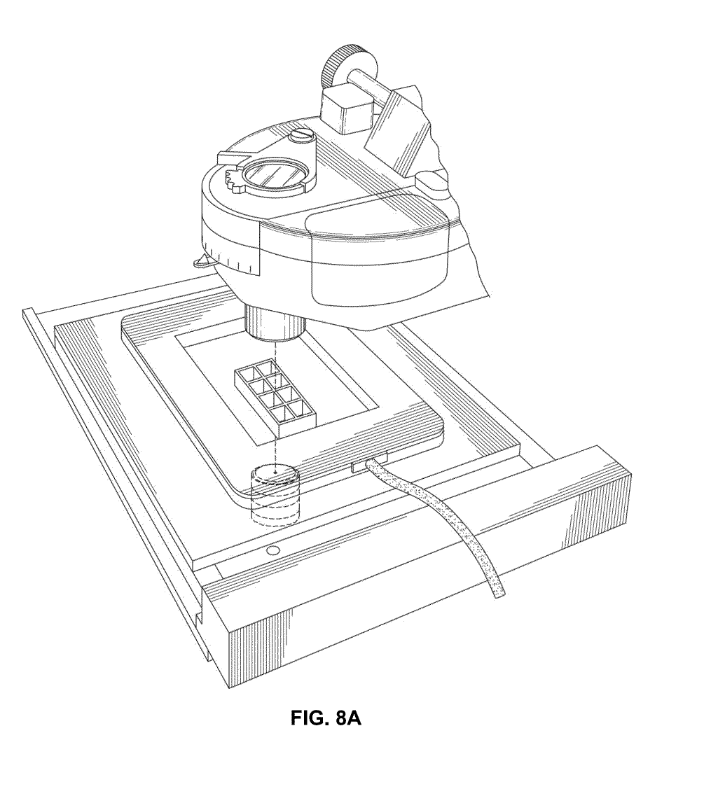

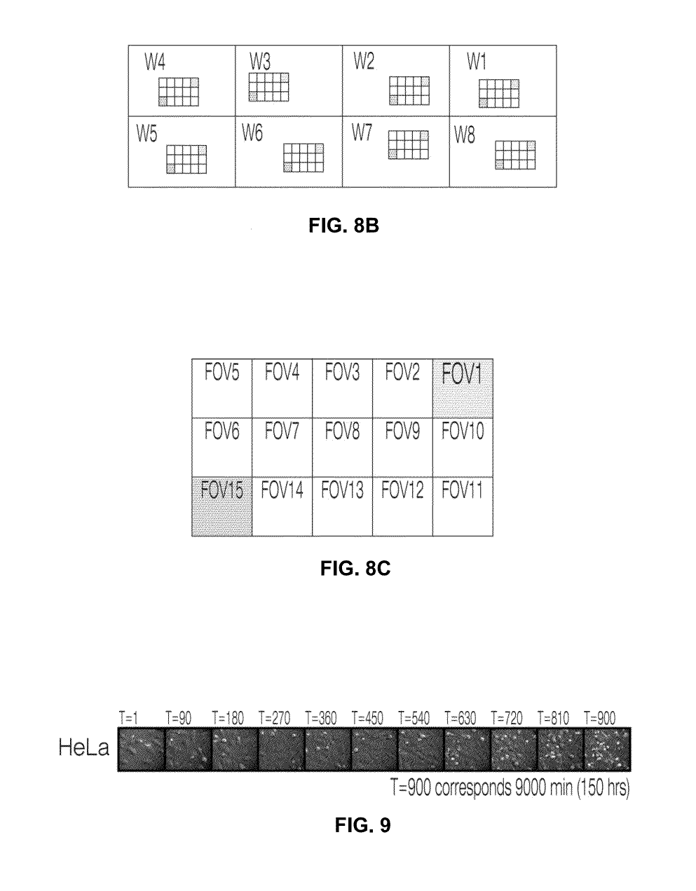

DIC microscopy, also known as Nomarski Interference Contrast (NIC) or Nomarski microscopy, is an optical microscopy illumination technique used to enhance the contrast in unstained, transparent samples. DIC works on the principle of interferometry to gain information about the optical path length of the sample, to see otherwise invisible features. A relatively complex lighting scheme produces an image with the object appearing black to white on a grey background. FIG. 8A is a graphical representation of the DIC microscope.

The image acquisition is controlled by software, which contains drivers for light source, shutters, filters, CCD camera, the X-Y stage and the Z-drives or others which are necessary to obtain images of cells. The software also has ability to generate raw image data files from at least 100 of multiple fields of views every 10 min or less with multiple z-stacks, for example 20 z-stacks. Any types of software, such as, for example, Metamorph or Volocity, which can drive the microscope system can be used.

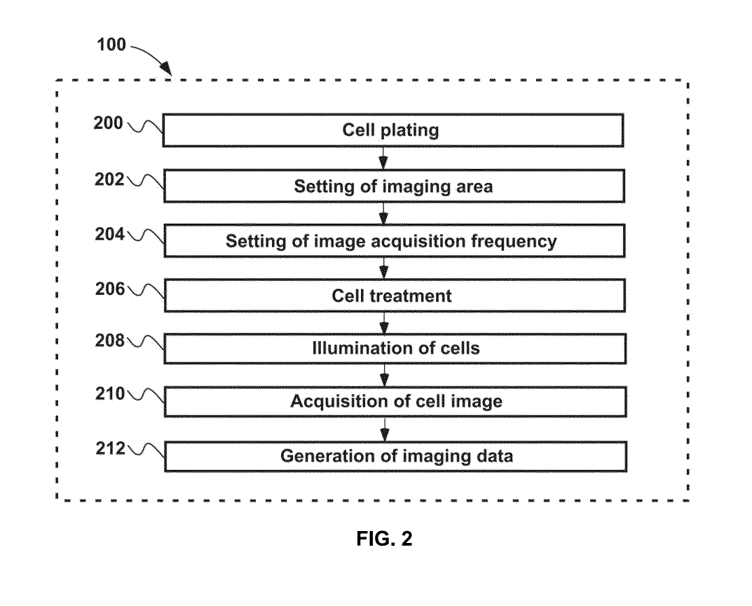

FIG. 2 shows a flowchart of this step. The order of steps 202, 204 and 206 can be changed. This process starts by the Cell plating 200. A certain number of cells, e.g. 5000 cells per one well of 8-well chamber, is plated into each well of multiwell chamber. Cells can be primary cultured mammalian cells, its derivatives, established mammalian cells, its derivatives or any cells that can be cultured and maintained in the wells. The Selection of imaging area 202 is carried out in order to find an area, which contains desired number of cells in a certain square micron meters. The number of cells can be any number of cells that fit to a well. Particularly the number of cells can be 100 cells or more; more particularly 1000 cells or more; particularly 10000 cells or more. If the image of the square micron meter cannot be obtained by one image acquisition, multiple field of views are arranged to cover the area. FIG. 8B is a graphical representation of the wells of the chambered coverglass. FIG. 8C is a graphical representation of the field-of-views of the wells of the chambered coverglass. The image acquisition can be controlled to acquire images of each field-of-view in each well at a predetermined frequency. In the example embodiment of FIG. 8, there are 15 field of views per well and 8 wells in the chambered coverglass, for a total of 120 field of views. Therefore, the stage could be moved to allow acquisition of the image of the first field of view (FOV1) of the first well (W1), to the last field of view (FOV15) of W1 and on to FOV1 of the second well (W2), all the way to FOV15 of the last well (W8) and then back to FOV1 of W1. The image capture can be at a rate of every 10 minutes for each field of view.

The Setting of the image acquisition frequency 204 is determined based on the mobility of cells. At the frequency for the image acquisition, a position of majority of cells in one image overlaps with the same cells in the next image. Because the mobility of each cell can be changed time to time and from cell to cell, the required % of overlap can be varied. In the Cell treatment 206, cells in each wells are either non-treated or treated with chemical compounds, inorganic substances, ionizing radiations, lights, magnetic fields, biological materials, including proteins/enzymes, factors, nucleic acids, glycans, virus, bacteria and parasites, metals ions and/or particles. Cells can be pre-exposed to reagents, e.g. for siRNA transfection, or factors prior the treatment. In some cases, different types of cells can be used. One well can serve as a control. In the Illumination of cells 208, to minimize light-derived phototoxicity caused by ultra violet light or emission light from a fluorophores exited by their appropriate wavelength of light, cells are illuminated by light with wavelengths, which are non- or less toxic for cells. Visible wavelength or near infrared are used for instance.

The Acquisition of cell image 210 is carried out by a way, which allows visualizing cellular structures, including nucleolus, nucleoli, mitochondria, cell granules, cell peripheries, outline of cells, shadow created by cells and/or light-reflecting parts of cells. At each field of view, images of planes at various depths within the sample (referred as to z-stacks) are taken to capture from the top to the bottom part of cells. The number of z-stacks is depending on the height of cells and the extent of focal plane drift, which often occurs during the long-term imaging of cells. Thus, minimal number of z-stacks is 1 and the 21 z-stacks are taken for HeLa cells for instance. The Generation of imaging data 212 creates gray scale images, which have graphic format of e.g. TIFF, JPEG, EPS, PICT, or BMP.

The Image quality controller 102 receives image files from the Image acquisition unit 100. After adjustment of contrast, selection of focused images, if multiple images acquired from each field of view are needed to be combined into one image file, these images are stitched. Resulting image files are transferred to the Movie creator 104.

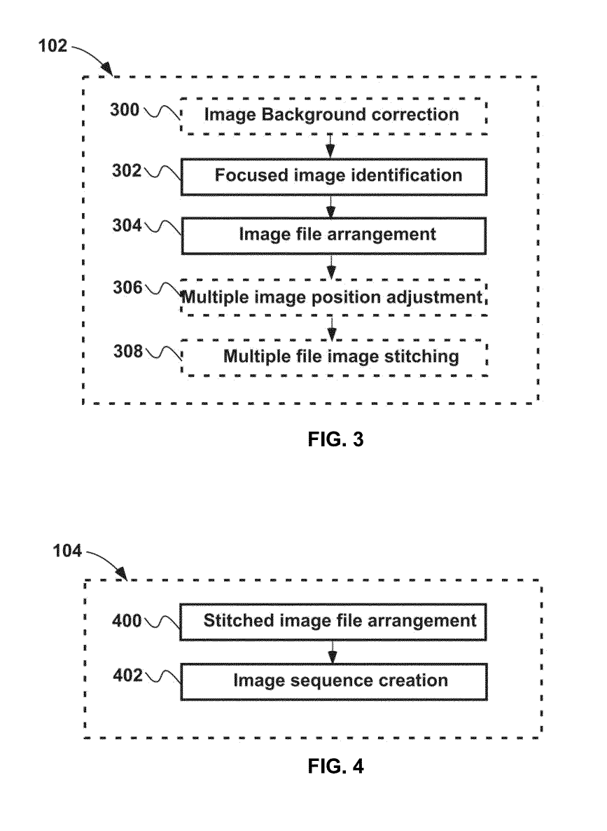

FIG. 3 shows a flowchart of this step. The order of steps 300, 302 and 304 can be changed. Steps indicated by dotted box can be skipped if these steps are not required. The Image background correction 300 carries out by subtraction of background images from images created by the Image acquisition unit 100. The background images can be ones prepared by image acquisition of corresponding multiwell chamber, which does not contain cells. Then, image contrast is adjusted by setting appropriate mean value and lower and higher threshold value of gray scale images. The Focused image identification 302 selects the best-focused or the images closed to the best-focused ones among multiple z-stacks, which cover top-to-bottom part of cells. If multiple fields of views are set by the Selection of imaging area 202, the image file arrangement 304 and the Image position adjustment 306 positions the images, which correspond to all fields of views, based on the X-Y position data recorded by image acquisition software. Then, positions of the image are adjusted. If X-Y position data is not available, image position adjustment can be carried out manually. The Multiple image stitching 308 receives adjusted X-Y position data from the Image position adjustment 306 and merges individual images into one image file.

The Movie creator 104 receives imaging files from the Image quality controller 102 and arranges files into an image sequence for movie.

FIG. 4 shows a flowchart of this step. The Stitched image file arrangement 400 organizes image files based on the well numbers of the multiwell chamber. The Image sequence creation 402 orders the stitched image files following the order of image acquisition to create an image sequence. Image sequence file number, which starts from 1, is assigned to each image file.

The Live cell events analyzer 106 receives image sequence files from the Movie creator 104. Among sequenced image files created by the Image sequence creation 402, an image file, which is designated as the Time point 1, is selected. The Time point 1 can be the image, which is acquired immediately after the Cell treatment 206. Thus, the Time point 1 can be the Image sequence file 10 for instance. Following Image sequence files can be Time point 2, Time point 3 etc. Then, cell lineage numbers are assigned to cells in the image of the Time point 1. Cells in the Time point 1 are designated as Progenitors. Each Progenitor and its progenies are tracked. Cellular structures, which are recorded by the Acquisition of cell image 210, are used as markers to track cells. The tracking can continue until to the Time point End, which is the last image files used for cell tracking. During the tracking, if normal and distinctive cellular events occur, these events are indexed. Then the Live cell events analyzer 106 creates a database, which contains information of cells, e.g. the indexed data and Time point number and X-Y position of cells.

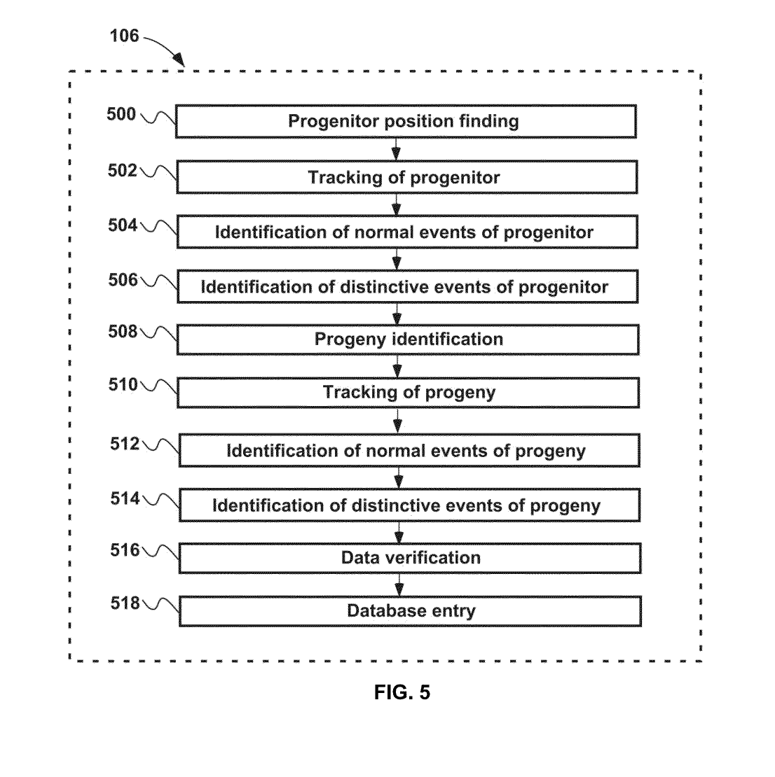

FIG. 5 shows a flowchart of this step. Order of Steps 502, 504 and 506, and Steps of 508, 510 and 512 can be changed. Steps 502, 504 and 506 apply to all Progenitors. Steps 508, 510 and 512 apply to all progenies. The Progenitor position finding 500 assigned cell lineage numbers to all or part of cells found in the Time point 1. The cell lineage number associates with X-Y position of cells. In addition to cell lineage number, cell number is assigned and the number of Progenitor is “0”. During the process of the Tracking of progenitor 502, X-Y positions of each cell in the following Time point are determined. If cells move out of the image, the cells are marked with “out of frame” or OF. If there are cells which move into an image, these cells can remain untracked or can alternatively be tracked. The Identification of normal events of progenitor 504 indexes normal cellular events. Such events include mitosis (M) and dipolar cell division (DD). Indexed data associates with the Time point number and X-Y position of cells. The Identification of distinctive cellular events of progenitor 506 indexes distinctive cellular events. Such events include tripolar cell division (TD), tetrapolar cell division (QD), quadpolar cell division (HD), cell fusion (CF), cell death (CD) and impaired cell division (IP). Each distinctive cellular event can be subdivided if more precise information is required. For example, CD can be sub-indexed by mitotic catastrophe and cell death occurred during G1 phase. Indexed data associates with the Time point number and X-Y position of cells. If the events lead to the cell division, created progenies are identified by Progeny identification 508. The cells numbers are assigned to these progenies. The events, which can lead to the formation of progenies, are DD, TD, QD and HD. Tracking of progeny 510, Identification of normal events of progeny 512 and Identification of distinctive cellular events of progeny 514 are equivalent to the Steps 502, 504 and 506 of Progenitors. If the progenies produced their own progenies, Steps 508, 510, 512 and 514 are carried out for the progenies. These steps can be repeated until image reached to the Time point End. After tracking of a progenitor and all of its progenies, X-Y position, indexed data and information for the linking of the Progenitor to all of its progenies are verified by the Data verification 516. If errors are identified, relevant Steps, e.g. Steps 502, 504 and 506 and/or Steps 508, 510, 512 and 514, are repeated. Such errors can be loss of cell tracking and mixing up one cell to another. Resulting data is entered into a database by the Database entry 518. The database contains cell lineage number, cell number, X-Y position, Time point number, indexed event and information, which determine the relationship of each cell.

Distinctive cellular events also include the following: Cell shape alteration is the modification of the overall shape of the cell during observation. Nuclear shape alteration is the modification of the shape of the nucleus of the cell during observation. Inner material accumulation is when a cell contains at least one other cell or engulfs a foreign material such as a microorganism or a particle or forms a structure composed by a protein, a nucleic acid and a lipid. Cell enlargement is when the size of the cell grows during observation without yielding progenies. Hyper-mobilization is when a cell migrates more than the average mobility range for the majority of cells, for example the cell may migrate 200% more than the average mobility rate, Hypo-mobilization is when a cell migrates less than the average mobility rate for the majority of cells, for example the cell may migrate 30% less than the average mobility rate. In another case, a cell could become attached to or could “cuddle” a neighboring cell if its/their mobility rate is low enough. This would also be hypo-mobilization. Prolonged doubling time is when the time between one division and the next division for a particular cell is for example 50% longer than the average time of the majority of cells.

The Cell lineage creator and data processor 108 receives data, which are entered into database by the Live cell events analyzer 106. Time point number, indexed events and data indicating relationships of one cell to other cells are used to create cell lineage maps. Various parameters, for example, cell growth rate, doubling time and frequency of cell death, cell fusion and abnormal cell division are determined. By mathematical and statistical calculation by applying certain biases to particular indexed events or doubling time, characteristics of distinctive and/or normal events of cell population are also determined. Analyzed data are entered into master database and data, which are already entered into the database, are used to evaluate the effect of treatment on cells.

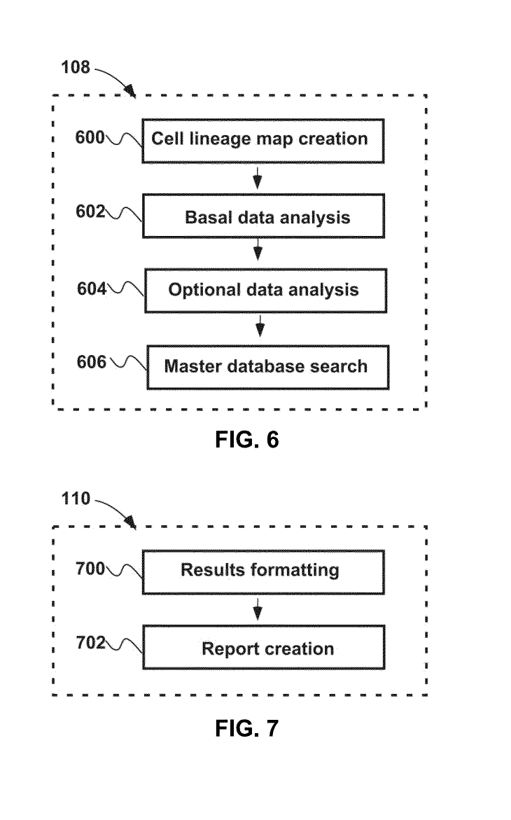

FIG. 6 shows a flowchart of this step. The Cell lineage map creation 600 collects the cell lineage numbers, cell numbers, Time point numbers and indexed event data of Progenitors and their progenies from the database generated by The Live cell events analyzer 106. Cell numbers are used to determine the drawing order of each cell. The Basic data analysis 602 analyze cell growth rate, doubling time and the frequency of cell death, cell fusion, normal cell division and abnormal cell division. Other event types can also be included into this basic data analysis. The Optional data analysis 604 performs mathematical calculation by applying certain biases to particular events and/or statistical analysis. For example, an index can be calculated by the formula of 1−number of DD*1+number of TD*0.8+number of QD*0.8+number of HD*0.8−number of CD*0.1−number of CF*0.1−number of IP*0.1. Resulting index value can be used to evaluate viability or individuality of cells derived from a progenitor. Different formula and constants can be used for the calculation. Resulting analyzed data are entered into the master database. Data in the database can be organized by cell type, treatment and/or dose. The master database search 606 collects relevant data from the master database in order to evaluate the effect of treatment on cells.

The Reports generator 110 generates results.

FIG. 7 shows a flowchart of this step. The Results formatting 700 organizes results in the form of Table and Figures. The Report creation 702 creates report, which can be displayed on computer monitor or printed.

The period of time for which the cells are under observation should be long enough for at least one progenitor to divide into progenies. For example, the period of time could be 7 minutes, 100 minutes, 160 hours, 330 hours, one month, etc.

### Example Microscope System

Different microscope systems can be designed for use in the present system. The following description is for an example microscope system.

The cells should be kept in condition for optimal cell viability and uncorrupted cell division. The microscope system should facilitate tracking of large numbers of individual cells. In some embodiments, different subsets of cells are imaged in parallel under the same time frame and conditions. Image quality and sampling frequency should be of sufficient quality to allow identification of detailed cell features required for automated tracking of cell division events and cell viability.

Differential Interference Contrast (DIC) with near infrared (NIR) light (>700 nm) provides the best prophylactic lighting conditions and required resolution for continuous cell imaging. DIC imaging provides sufficient detail to track cell behavior while providing high contrast data for computer analysis. The microscope system uses Back thinned Electron Multiplication Cameras (BT EMCCD).

An example microscope system can use a high resolution nonimmersion objective which gives enough working distance to freely move between different sample populations and also eliminates the concern of focal drift and cell viability due to localized temperature fluctuations. A magnification system compensates for a small loss in resolution.

The heated housing area for the cells is required to accommodate triple gas or single gas perfusion and humidity control for a multichambered cell well system designed around optic grade glass.

### Example Tracking Software

Different tracking software routines can be designed to automate the tracking portion of the process. The following are example routines for carrying out the tracking and classifying (identification) of the cells and cell events.

The auto-cell tracking system employs gravity center tracking and non-fluorescence image processing and can be summarized as follows: 1. Apply Gaussian bluer to remove noise. 2. Apply threshold and then paint the area above the threshold (bright parts of cells are extracted). 3. Carry out connectivity analysis. 4. Identify the connective pixels of the target cell. 5. Determine the gravity center of the connected pixels. 6. Load next image. 7. Repeat step 1-5. Gravity center indicates the position of the target cell. Thus this software is capable to track cells.

If one neighboring mitotic cell moves close to the target cell, two connective pixels merge. Thus, this software is no longer able to segment two cells. Because the gravity center of the target cell shifts due to the merging, when this program detects the shift, it terminates cell tracking.

The cell division identification works as follows: 1. Up to the connectivity analysis process, the same approach as described above is used. 2. Draw vectors around the connective pixels. 3. Create vector pattern library (the library contains about 100 repetitive patterns). 4. Compare vector pattern of the target cell with the patterns in the library. If the pattern of target cell matches with any one in the library, the cell is considered to enter mitosis. 5. Determine the connected pixel number of the mitotic cell and its gravity center. 6. Load next image. 7. Check the gravity center of the mitotic cell and the number of pixels of the connective pixels. If the pixel number is significantly reduced (˜40-60%), the software recognizes that cell division occurs. 8. Look for the nearest connected pixels to find its sibling. This program works for most dipolar cell divisions.

If the pixel number reduction is less than 40% or more than 60%, auto-cell tracking is terminated as either non-typical dipolar or tripolar cell division occurs.

Reinstallation of manual cell tracking can be done for terminated processes.

To track the movement of mitotic cells close to target cells, the steps of the analysis software are the following: 1. In order to recognize an approaching cell, gravity center of all connective pixels in the image will be determined. 2. If the approaching cell moves on the target cell, connective pixels of the approaching cell will be merged with the connective pixels of target one, implying that one gravity center will disappear. This is the signal to start the handling of the “moving of mitotic cells close to target cell” situation. 3. The mitotic cell is usually brighter than the target cell, as it reflects more light. Position of the mitotic cell is predicted by carrying out connectivity analysis of brighter pixels. 4. Software focuses on connective pixels of both brighter and remaining pixels (determines gravity center of each ones). 5. When the connective pixel of brighter pixels starts to move, the software tries to segment the target cell. This can be done by applying several different thresholds to the image and carrying out connectivity analysis. If target cells can be segmented, the process comes back to normal tracking

For cell division, the software checks the images as follows. 1. When the target cell enters mitosis, determine the gravity center of connective pixel of the cell and, if the software failed to detect cell division, load the next image. 2. Because divided cells are created near the position of its mitotic cell, software searches connectivity pixels around that position and, if software finds a connectivity pixels, examine whether it belongs to non-target cells. 3. If it does not belong to non-target cells, the connectivity pixels will be marked as a candidate for progeny of the mitotic cell. 4. Load next image and repeat step 2-3. Continue this process until two (dipolar division) or three (tripolar division) connective pixels are identified.

### Usage Examples

The apparatus described herein can be used to monitor the individuality of cells in a cell population to track distinctive behavior. For example, this can be used in a quality control context where a biological product is tested before being injected in a patient to ensure that the cell population to be injected does not contain individual cells displaying distinctive behavior with respect to what is expected of the cell population in general.

The apparatus described herein can also be used to compare the reaction of a cell population to a treatment. For example, two identical cell populations can be placed in different wells of the chamber of the apparatus and kept under the same environmental conditions. One cell population is treated with a treatment and the other is kept as a control population. The individuality of the cells in both wells is tracked to note distinctive behavior. Comparisons between the noted distinctive events of the treated population with respect to the control population can yield interesting conclusions on the effect of the treatment on the cell population.

### Example Experiment

HeLa cells were purchased from ATCC. HeLa cells (1×10⁴ cells in 50 μl per well) were carefully plated in the center of a coverglass Lab-Tek 8 wells chamber for optimal optical observation. After cells were stably attached, 500 μl of culture medium was added. HeLa cells were used after 24 hrs of plating. Cultures were maintained until cells occupied over 90% of the surface of each chamber.

By employing a 8 well-chambered coverglass, cells treated with various doses of substances can be monitored simultaneously, allowing real-time cell biological assays to be performed on the microscope stage with their appropriate control (FIG. 8).

The HeLa cells showed optimal mobility since the cells moved around an area of about 10 to 50 μm diameter. Furthermore, their reasonable mobility decreased the chance of pilling up of HeLa cells, allowing precise individual cell tracking.

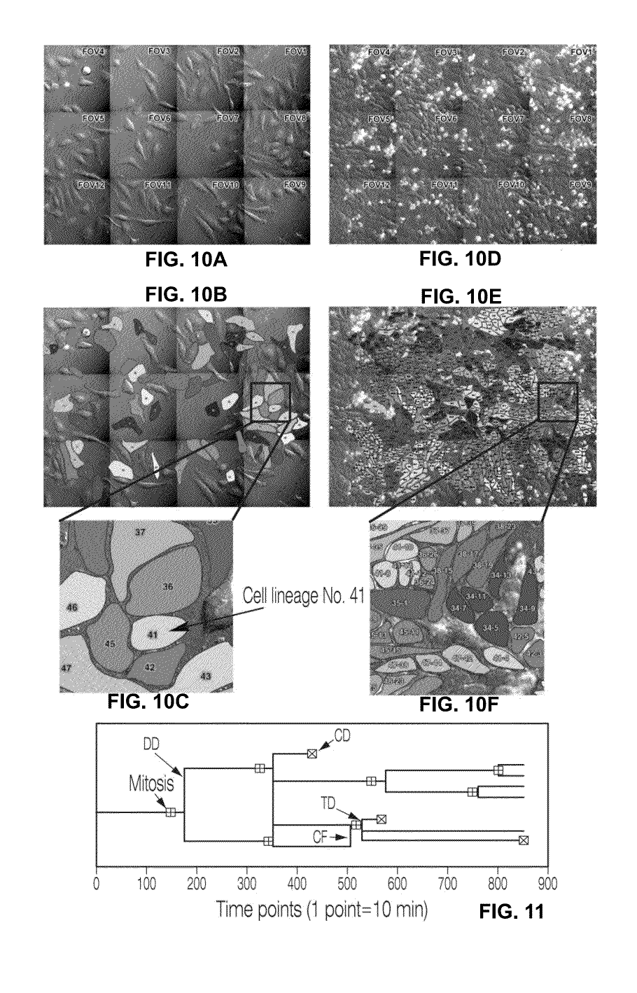

A Quorum WaveFX Spinning Disc Confocal System (Quorum Technologies Inc., Canada) with a Leica microscope controlled by Volocity v4.0 was employed for long-term live cell imaging. In order to eliminate any risk of phototoxicity, differential interference contrast (DIC) images were taken through HCX PL APO 40× oil objectives (NA=1.25) by using a Halogen lamp as a light source (UV light from the lamp was removed nearly to 100% using DIC prism filters). Cells grown on a coverglass Lab-Tek 8 well chamber were then placed on the microscope stage and were cultured using an environmental chamber at 37° C. with 7.5% humidified CO₂ (LiveCell™ system, Pathology Devices Inc, MD). XY positions of panoramic fields of views (FOVs) were then registered using Volocity v4.0. Typically, 4×3 or 4×4 FOVs were used for HeLa cells. In order to minimize the bias caused by variations in initial cell density, typical panorama views (see an example of a panorama view in FIG. 10A) were printed and used as reference for the determination of the position of FOVs. Focus adjustment of each FOV was then carried out. DIC images were captured every 10 min (34 msec exposure for each plane of FOV) from the +10 to the −10 μm position relative to the focal plane with 1 μm increment using a piezo focus drive. At each FOV, 21 z-plans were thus created. In each well, the microscope stage moved from FOV1 of well 1 (W1) to FOV15 of well 8 (W8). Total number of FOVs was 120.

FIG. 9 is a sequence of images extracted from the images taken at different times to show the progression in the quantity of cells present in one field-of-view.

During the typical cell plating, for example preparing cell suspension and plating a known number of cells to a culture dish or a well of a multiwell chamber, cells often accumulate on the periphery or the center of dishes or wells due to subtle differences in the flatness of the bottom of the chamber. Although such an accumulation could create high and low cell density areas within a dish or a well, local variations in cell density have been considered as having a negligible impact on the majority of cell biological studies. However, the growth rate and perhaps the response of cells to a treatment could differ between high and low-density areas. Thus, in order to obtain reproducible results, we selected FOVs for which the density is about 60-70% for non-treated HeLa cells.

siRNA treatment was carried out after placing a coverglass Lab-Tek 8 well chamber on the microscope stage. Scramble siRNA as control (2 μg, medium length, Invitrogen) or p53 siRNA (2 μg, New England Bio Labs) were mixed with 8 μl EC buffer and 0.4 μl enhancer reagents (Effecten kit, Qiagen) for 5 min. Then 1 μl effectante (Qiagen) was added to the mix followed, after 10 min of incubation, by 120 μl of culture medium. To each well, 118 μl of mixture was added and cells were cultured for 24 hrs in the environment chamber on the microscope. After replacing the mixture with complete medium, cells were cultured another 24 hrs and then were exposed to methylnitronitrosoguanidine (MNNG). Because transfected cells contained lipid vesicles, transfection efficiency was estimated through visual examination and efficiency was concluded to be over 99%. Transfection efficiency using Cy3-conjugated siRNA was also 98% and the expression level of p53 detected by anti-p53 antibody (Calbiochem) was reduced to less than 10% of control 48 hrs after transfection (data not shown).

After initiation of cell monitoring, the location of FOVs cannot be changed. Thus, in order to carry out long-term cell imaging that would cover more than 100 cell lineages through the entire observation period, we selected an area that contained the appropriate number of cells. In the case of HeLa control cells or cells exposed to non-cytotoxic doses of MNNG, an area of about 70% surface area occupancy was selected. Because HeLa cells were capable to move around in a 10-50 μm diameter, most of HeLa cells could find free culture surface, allowing at least 5 to 6 cell divisions within 160 hrs.

Volocity image sequence files (multi-layer TIFF) were split into single-layer TIFF files. Splitted images were displayed using the Volocity v4.0 program in order to visually determine focal planes. The selected focal plane was then moved into a folder created within Volocity v4.0. Images containing focal planes were exported as TIFF files. Movies were created by employing QuickTime player Pro. If image quality was not optimal for cell tracking, contrast of TIFF images were adjusted using the batch processing function of Photoshop v7.0.

Cells were concurrently monitored using DIC images for 100˜160 hrs to detect low frequency cellular events. In a typical experiment, cell monitoring was done using 120 panoramic FOVs, (see FIG. 8) for 160 hrs, creating 2,268,000 image files, which were eventually converted into 120 independent imaging movies.

Cell tracking was carried out. A panorama view of every well was prepared (see FIG. 10A) in order to assign a unique cell lineage number (we defined cell lineage as a group of cells derived from a progenitor identified at T=1) to each cell being followed by individual cell tracking (see FIGS. 10B and 10C). During this assignment, all cells were included to perform analysis in an objective manner. After assigning cell lineage numbers, time point of mitosis, cell division, cell fusion and cell death were determined based on cell morphology and were entered into the Database. For mitosis, initial time points were an indication that the event occurred as it is difficult to precisely pinpoint using morphological observations. Similarly, initial time points for cell fusion events, particularly between F1 (progenies of a progenitor cell) and F2 (second-level progenies of a progenitor cell), were an indication of fusion events, as cell fusion often takes place gradually. To reduce tracking errors, most of the tracking was reconfirmed once by rewinding the movie.

Upon creation of cell lineage maps, a unique identification number was assigned to each progeny (see FIG. 10E for an example result of individual cell tracking and see FIG. 10F for an example numbering of progenies). Using this numbering, images of tracked cells can be retrieved from the live cell imaging movies.

Time points for events determined during cell tracking were entered into the Database.

FIG. 11 is a graphical representation of a cell lineage diagram based on data found in FIG. 10, this map corresponds to cell lineage 41 indicated in FIG. 10C. During long-term live cell imaging, progenies entered cell death (CD), dipolar division (DD), tripolar division (TD) and cell fusion (CF).

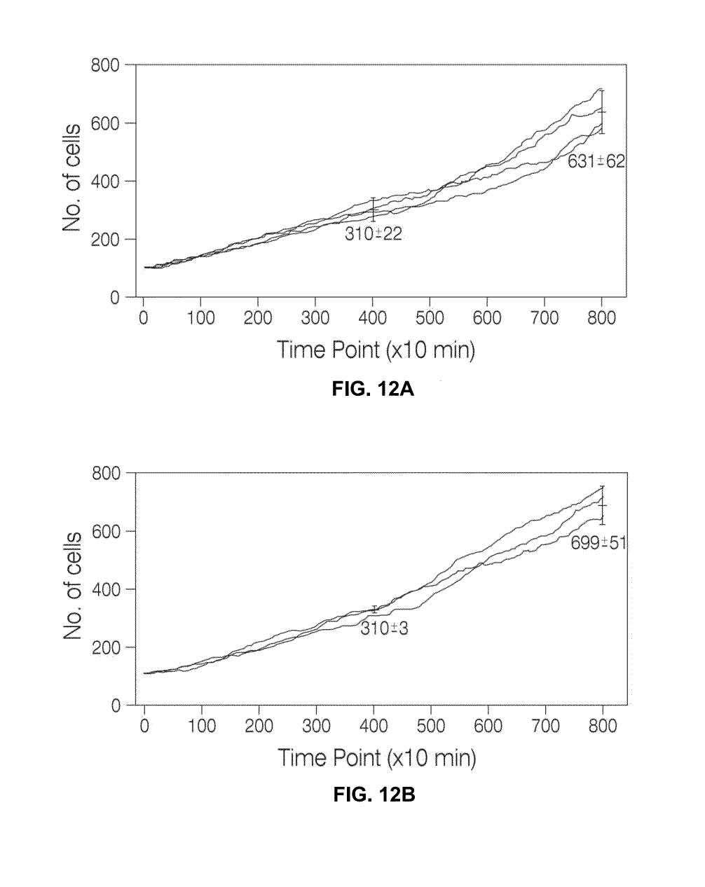

Numbers of cells for each 10 min period extracted from the database of the four independent control experiments and analyses were plotted and results are shown in FIG. 12A which include individual cell tracking, cell lineage map creation and data analysis to determine cell growth rate. Mean and standard deviation at the 4000 min point were 301 and 22, respectively. Although the standard deviation was increased at the 8000 min point, it is still within the 10% of mean value, suggesting that the system generates reproducible results.

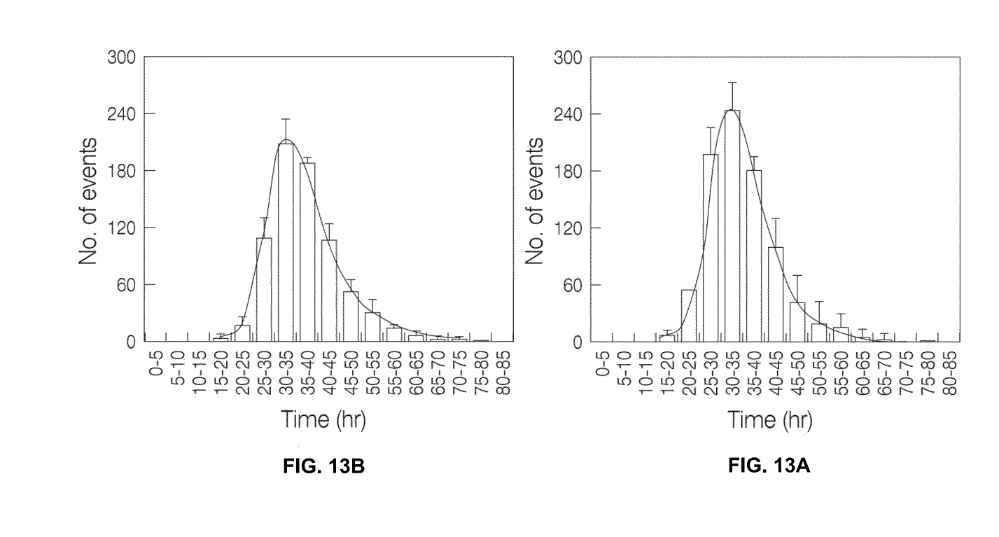

We found that 1 μM MNNG did not show any effect on cell growth rate (FIG. 12B). In addition, no major prolongation of doubling time in cells exposed to 1 μM MNNG was found (FIGS. 13A and 13B), indicating that cell growth was not perturbed by this dose of MNNG. In addition, the number of dead cells found in the cell culture treated with 1 μM MNNG was almost similar to that of control (Table 1, CD). Therefore, we concluded that 1 μM MNNG is a non-cytotoxic dose for HeLa cells.

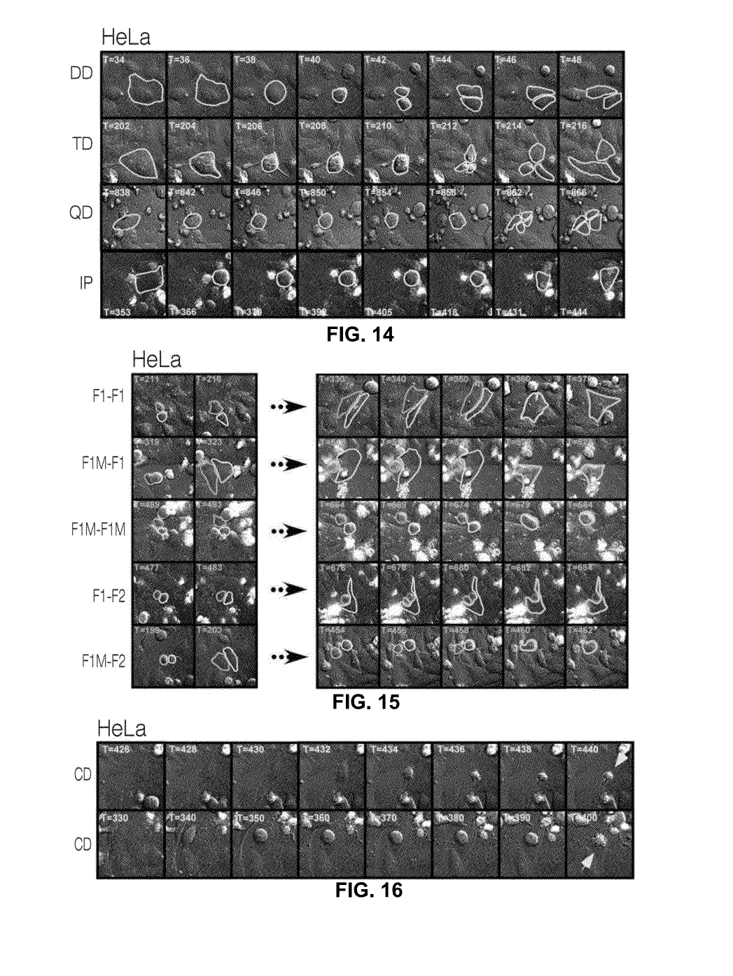

FIG. 14 includes photographs showing the categorization of dipolar division (DD), tripolar division (TD), quadrupolar division (QD) and impartial mitosis (IP) for HeLa cells.

FIG. 15 includes photographs showing the categorization of cell fusion (CF), in which HeLa cells in T=1 were defined as F0 and first-level progenies and second-level progenies as F1 and F2, respectively, F1M represents progenies that entered mitosis.

FIG. 16 includes photographs showing the categorization of cell death (CD) for HeLa cells, including all types of CD regardless of observed patterns which depend on cell types.

Table 1 shows a Summary of distinctive cellular events (HeLa). A quantity of 120 to 200 progenitor cells was tracked. Data were normalized by 200 progenitors. The legend for Table 1 is as follows: a. Growth folds were calculated by dividing a cell number at T=900 by cell number at T=1, b. Total cell division (DV) is sum number of cell division events; total DV=dipolar division (No.)+tripolar division (No.)+quadrupolar division (No.), c. Multipolar division (MD) is sum number of MD events; tripolar division (No.)+quadrupolar division (No.), d. Cell fusion (CF) is sum number of CF events; Total CF=(F1-F1)+(F1-F1M)+(F1MF1M)+(F1-F2)+(F1M-F2)+(F2-F2). Cells in T=1 was defined as F0 and first-level progenies and second-level progenies as, F1 and F2, respectively. F1M represents progenies that entered mitosis, e. CD: Cell death, p values were calculated by comparison to the events occurring in non-treated cells, *: P<0.05, **: P<0.1.

TABLE 1
Summary of distinctive cellular events (HeLa)

| Types of | Non-treated | Non-treated | MNNG 1 μM | MNNG 1 μM |
|---|---|---|---|---|
| events | Total No. | % of Total DV | Total No. | % of Total DV |
| Growth | 6.3 ± 0.6 | | 7.0 ± 0.5 | |
| fold^{a} | | | | |
| Total DV^{b} | 1716 ± 220 | | 1983 ± 199 | |
| MD^{c} | 53.2 ± 8.8 | 3.1 ± 0.8 | 88.3 ± 17.5** | 4.5 ± 0.2** |
| CF^{d} | 115.5 ± 11.1 | 6.6 ± 0.2 | 149.3 ± 25.6* | 6.3 ± 2.3 |
| CD^{e} | 267.2 ± 34.2 | 28.7 ± 5.3 | 273.4 ± 49.1 | 26.1 ± 4.2 |

Then, to determine whether a non-cytotoxic dose of MNNG induces distinctive cellular events, e.g. multipolar division and cell fusion, we first determined the basal frequency of those events by employing database and cell lineage data obtained with non-treated HeLa cells. We found that non-treated HeLa cells entered multipolar division, namely tripolar division (TD) and cell fusion (CF). Results summarized in Table 1 show that the total number of multipolar division and cell fusion (CF) events was 53 and 115, respectively. In addition, about 73% of multipolar division events occurred following cell fusion. In this case, two cells remained linked, fused and then entered tripolar division. However, in a significant number of cases, such links were not clear. When database and cell lineage data obtained with cells exposed to non-cytotoxic dose of MNNG were analyzed, a total of 88 multipolar events, which was about 1.7 times higher than for non-treated cells, were found. We also found that the non-cytotoxic dose of MNNG has a slight promoting effect on HeLa cell growth (Table 1). Thus, % of total cell division was calculated in order to normalize this effect. Even after such normalization, statistically significant difference in % of multipolar division events was also found for cells exposed to a non-cytotoxic dose of MNNG when compared to non-treated cells. On the other hand, in the case of cell fusion, % of total cell division events was not changed (Table 1), although total number of cell fusion events was slightly increased by the exposure of HeLa cells to the non-cytotoxic dose of MNNG. These results indicate that the total number of cell fusion events was increased proportionally to the total number of cell divisions, while the non-cytotoxic dose of MNNG showed significant promoting effect on the risk of cells entering multipolar divisions.

Our results suggest that, although a non-cytotoxic dose of MNNG did not show any detectable effects on cell growth rate and doubling time of HeLa cells, the risk of these cells entering multipolar division was increased by the exposure. Because such events occurred in a handful of cells among a vast majority of normal or non-affected ones, they have been difficult to detect by currently existing methods.

While illustrated in the block diagrams as groups of discrete components communicating with each other via distinct data signal connections, it will be understood by those skilled in the art that the illustrated embodiments may be provided by a combination of hardware and software components, with some components being implemented by a given function or operation of a hardware or software system, and many of the data paths illustrated being implemented by data communication within a computer application or operating system. The structure illustrated is thus provided for efficiency of teaching the described embodiment.

The embodiments described above are intended to be exemplary only. The scope of the invention is therefore intended to be limited solely by the appended claims.

We claim:

1. A method for quantitative identification of distinctive cellular events occurring in a cell population using a non-fluorescence approach, the method comprising:
providing an image acquisition unit having a Differential Interference Contrast microscope system with a camera, a light source, an environmental chamber allowing carrying out cell culture of at least one cell in a cell population, for a period of time sufficient to allow division of a progenitor to form at least one progeny;
controlling the image acquisition unit to acquire images of said cell population in the chamber, at predetermined time points;
individually tracking said at least one cell of said cell population in said images acquired by said image acquisition unit;
detecting an occurrence of a distinctive cellular event for at least one of said cell individually tracked by said cell tracker; and
generating a report having an identification of detected distinctive cellular events,
wherein said distinctive cellular event is selected from the group consisting of: dipolar cell division (DD), tripolar cell division (TD), tetrapolar cell division (QD), quadpolar cell division (HD), cell fusion (CF), cell death (CD), impaired cell division (IP), cell shape alteration, nuclear shape alteration, inner cellular material accumulation, cell enlargement, engulfing, hyper-mobilization, hypo-mobilization and prolonged doubling time, and combinations thereof.

2. The method of claim 1 wherein the tracking of the at least one cell of the cell population includes gravity center tracking including assigning an X-Y position of the at least one cell in the cell population at predetermined time points, and assigning a cell lineage number to each of the at least one cell in the cell population.

3. The method of claim 2 wherein the report includes cell lineage information of each of the at least one cell in the cell population, wherein the cell lineage information is based on the cell lineage number, the X-Y position, and distinctive cellular events for each of the at least one cell in the cell population.

4. The method of claim 1 wherein the report includes cell lineage information.

5. The method of claim 1 wherein the distinctive cellular event is selected from the group consisting of: dipolar cell division (DD), tripolar cell division (TD), tetrapolar cell division (QD), quadpolar cell division (HD), cell fusion (CF), cell death (CD), impaired cell division (IP), and combinations thereof.

6. The method of claim 1 wherein the report includes an index, wherein the index is calculated by the following formula:
Index=(number of DD)+0.8(number of QD)+0.8(number of HD)−0.1(number of CD)−0.1(number of CF)−0.1(number of IP).

7. The method of claim 1 wherein the report includes a frequency of distinctive cellular events for the treated cells, wherein said frequency is indicative of carcinogenicity of the candidate carcinogenic compound.

8. The method of claim 1 wherein said cell population is treated with a non-cytotoxic dose of a substance.

9. A method of use of an apparatus for quantitative identification of rare distinctive cellular events occurring in a cell population using a non-fluorescence approach to detect an individuality of cells in said cell population, the apparatus comprising:
providing an image acquisition unit having a Differential Interference Contrast microscope system with a camera, a light source, an environmental chamber allowing carrying out cell culture of at least one cell in a cell population, for a period of time sufficient to allow division of a progenitor to form at least one progeny;
controlling, by a controller, the image acquisition unit to acquire images of said cell population in the chamber, at predetermined time points;
individually tracking, by a cell tracker, said at least one cell of said cell population in said images acquired by said image acquisition unit;
detecting, by a distinctive cellular event detector, an occurrence of a distinctive cellular event for at least one of said cell individually tracked by said cell tracker;
generating a report, by a report generator, wherein the report includes an identification of detected distinctive cellular events,
wherein said distinctive cellular event is selected from the group consisting of: dipolar cell division (DD), tripolar cell division (TD), tetrapolar cell division (QD), quadpolar cell division (HD), cell fusion (CF), cell death (CD), impaired cell division (IP), cell shape alteration, nuclear shape alteration, inner cellular material accumulation, cell enlargement, engulfing, hyper-mobilization, hypo-mobilization and prolonged doubling time, and combinations thereof.

10. The method of claim 9 wherein the individually tracking includes gravity center tracking that assigns an X-Y position of the at least one cell in the cell population at predetermined time points, wherein the cell tracker assigns a cell lineage number to each of the at least one cell in the cell population.

11. The method of claim 9 wherein the report includes cell lineage information of each of the at least one cell in the cell population, wherein the cell lineage information is based on the cell lineage number, the X-Y position, and distinctive cellular events for each of the at least one cell in the cell population.

12. The method of claim 9 wherein the report includes cell lineage information.

13. The method of claim 9 wherein the distinctive cellular event is selected from the group consisting of: dipolar cell division (DD), tripolar cell division (TD), tetrapolar cell division (QD), quadpolar cell division (HD), cell fusion (CF), cell death (CD), impaired cell division (IP), and combinations thereof.

14. The method of claim 9 wherein the report includes an index, wherein the index is calculated by the following formula:
Index=(number of DD)+0.8(number of QD)+0.8(number of HD)−0.1(number of CD)−0.1(number of CF)−0.1(number of IP).

15. The method of claim 9 wherein the report includes a frequency of distinctive cellular events for the treated cells, wherein said frequency is indicative of carcinogenicity of the candidate carcinogenic compound.

16. The method of claim 9 wherein said cell population is treated with a non-cytotoxic dose of a substance.
